(19) United States
(12) Patent Application Publication
(10) Pub. No.: US 2004/0249170 A1
(43) Pub. Date: Dec. 9, 2004

(54) Process for preparing an intermediate useful for the asymmetric synthesis of duloxetine

(76) Inventor: Alfio Borghese, Dreve Marechal Davoust (BE)

Correspondence Address: Arvie J Anderson, Eli Lilly & Company, Patent Division, PO Box 6288, Indianapolis, IN, 46206-6288, US

(21) Appl. No.: 10/500,829

(22) PCT Filed: Jan. 13, 2003
(86) PCT No.: PCT/US03/00018

Related U.S. Application Data
(60) Provisional application No. 60/351,622, filed on Jan. 24, 2002.

(51) Int. Cl. C07D 333/12; C07D 333/20

(52) U.S. Cl. 549/75

(57) ABSTRACT

This invention provides a process for the synthesis of(S)-3-Methylamino-1-2-thienyl)-1-propanol, a key intermediate in the synthesis of duloxetine.

### BACKGROUND OF THE INVENTION

This invention belongs to the fields of pharmaceutical chemistry and synthetic organic chemistry, and provides a process for the synthesis of a key intermediate in the preparation of duloxetine, (+) N-methyl-3(1-naphthalenyloxy)-3-(2-thienyl)propanamine, hydrochloric acid salt.

Duloxetine is a pharmaceutical now under development as an anti-depressant. It inhibits the uptake of both norepinephrine and serotonin and is presently in clinical evaluation. The compound was disclosed in U.S. Pat. Nos. 5,023,269 and 4,956,388 by Robertson, et al. and the synthesis of it was discussed in more detail by Berglund, R. A., Org. Proc. Res. Devel., 1, 328 (1997) and Deeter, et al., In Tetrahedron Letters, 31(40), 7101-04 (1990) and aspects patented in U.S. Pat. Nos. 5,362,886 and 5,491,243.

Synthetic schemes and processes have been reported for conversion to duloxetine. Two particular reported synthetic schemes in Liu, H.; Hoff, B. H.; Authonsen, T. Chirality, 12, 26 (2000) and Wheeler, W. J.; Kuo, F. S. Labelled Compd. Radiopharm., 36, 213 (1995), have a common chloroalcohol intermediate. In both cases this chloroalcohol intermediate is converted to an aminoalcohol, in two steps and then arylated to give duloxetine. These processes, as reported in the above articles, are outlined in Schemes 1 and 2.

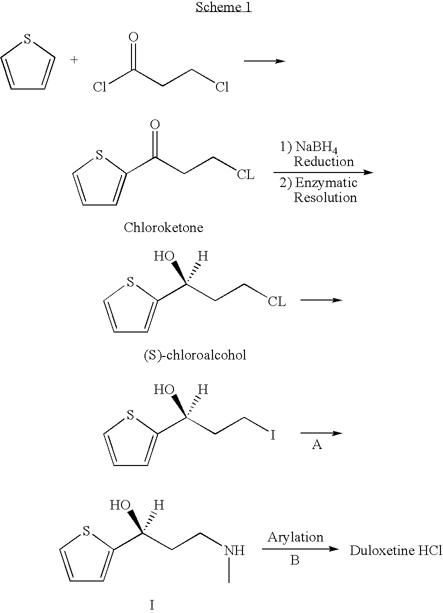

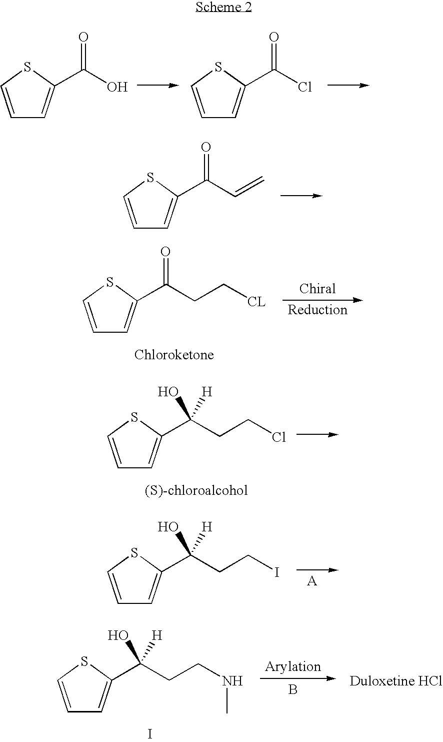

Although the arylation of (S)-3-Methylamino-1-(2-thienyl)-1-propanol, compound I in in Schemes 1 and 2, has been disclosed, its resolution via diastereomeric salt formation has not been shown. Further, while Racemic ((R/S)-3-Methylamino-1-(2-thienyl))-1-propanol has been disclosed in Bopp, R. J.; Kennedy, J. H., LC-GC, 5, 514 (1998), the resolution of this key intermediate has not been successful.

The present invention provides improved conditions for carrying out the resolution of ((R/S)-3-Methylamino-1-(2-thienyl))-1-propanol whereby resolved compound I is obtained in greater enantiomeric purity and yield than has previously been possible.

### SUMMARY OF THE INVENTION

The present invention provides a process for preparing (S)-(+)—N,N-dimethyl-3-(1-naphthalenyloxy)-3-(2-thienyl)-propanamine comprising resolving racemic ((S)-3-Methylamino-1-(2-thienyl))-1-propanol with 2,3,4,6-di-O-isopropylidene-2-keto-L-gulonic acid or S-(−)-2-pyrrolidone-5-carboxylic acid in a first organic solvent, and if desired, racemizing a stereomerically enriched mixture in an isopropanol/hydrochloric acid mixture; and if desired, crystallizing (S)-3-Methylamino-1-(2-thienyl)-1-propanol by resolving racemic ((R/S)-3-Methylamino-1-(2-thienyl))-1-propanol with 2,3,4,6-di-O-isopropylidene-2-keto-L-gulonic acid in a third organic solvent.

### DETAILED DESCRIPTION OF THE PREFERRED EMBODIMENT

The present invention provides a process for preparing the specific enantiomer shown above as compound I in Schemes 1 and 2 above. It is named (S)-3-Methylamino-1-(2-thienyl)-1-propanol.

The resolution step of the present invention is prepared by adding 1 molar equivalent of 2,3,4,6-di-O-isopropylidene-2-keto-L-gulonic acid or (S)-(−)-2-pyrolidinone-5-carboxylic acid, preferably 2,3,4,6-di-O-isopropylidene-2-keto-L-gulonic acid, to racemic (S)-3-Methylamino-1-(2-thienyl)-1-propanol in an organic solvent at room temperature, yielding after crystallization a mixture of diastereomeric salts. The organic solvent may be, for example, isopropanol, tetrahydrofuran, acetone or ethyl acetate. Isopropanol is the preferred solvent. If the resolution is performed as part of a process which later involves crystallization of(S)-3-methylamino-1-(2-thienyl)-1-propanol, the above solvent is a first organic solvent. Diastereomerically enriched crystals are obtained by additional crystallizations. Example 1, below, illustrates this procedure in detail. Chiral analysis was done by capillary electrophoresis(ce) and the results summarized in Table I below.

TABLE 1
Optical resolution and purification of racemic (S)-3-Methylamino-1-
(2-thienyl)-1-propanol in isopropanol.

| | Crystals | | | |
|---|---|---|---|---|
| | Enantiomeric | ML Enantiomeric | Cryst. | |
| | Composition | Composition | Yield | ML Yield |
| | (% area/% area) | (% area/% area) | (%) | (%) |
| Optical | 56/44 | 25/75 | 74 | 23 |
| resolution | | | | |
| 2^{nd} optical | 62/38 | 28/74 | 79 | 19 |
| purify. | | | | |
| 3^{rd} optical | 89/11 | 26/74 | 58 | 38 |
| purif. | | | | |

ML = mother liquor

The enriched diastereomer salts (diasteromeric excess (d.e.)=7.8%) were obtained with 33% overall yield. The optical resolution of(S)-3-Methylamino-1-(2-thienyl)-1-propanol (d.e. =78%) measured in methanol is [α]_{D}²⁵=−9.6 (MeOH, C=4.4). The sign and the value of this optical rotation compared with the literature value [α]_{D}²⁵=−12.5 (MeOH, C=4.4), Huiling Liu, Bard, Helge, Hoff and Thorleif Authorsen, Chirality, 12, 26-29 (2000) of the (S) enantiomer demonstrates that the wanted stereomer is obtained with the resolving agent 2,3,4,6-di-O-isopropylidene-2-keto-(-gulonic acid with an optical purity of 76.8%. Additional crystallization steps would be necessary to obtain optical pure (S)-3-methylamino-1-(2-thienyl)-1-1-propanyl.

The racemization step of (S)-3-Methylamino-1-(2-thienyl)-1-propanol is achieved by partially racemizing a stereomerically enriched mixture (d.e. =76%) of (S)-3-Methylamino-1-(2-thienyl)-1-propanol (as a salt form with 2,3,4,6-di-O-isopropylidene-2-keto-L-gulonic acid) in isopropanol/hydrochloric acid mixture at room temperature as shown in Example 2 below. This process or another acid catalysis can be used to recycle the mixture of salts of (S)-3-Methylamino-di-O-isopropylidene-2-keto-L-gulonic acid enriched in the unwanted stereoisomer.

Finally a second order asymmetric induced crystallization was achieved by performing the optical resolution of racemic (S)-3-Methylamino-1-2-thienyl)-1-propanol with 1 equivalent of 2,3,4,6,-di-O-isopropylidene-2-keto-L-gulonic acid in an organic solvent such as isopropanol, tetrahydrofuran, acetone or ethyl acetate, preferably isopropanol, at 40° C., and the reaction mixture left agitated at that temperature for 66 hours. If the crystallization is performed after the resolution step described above, the organic solvent used in the crystallization step is a second organic solvent. An example is provided below. The diastereomeric crystals were obtained with a yield of 76% (diastereomeric composition (−)/(+)=88%/12%); (M. L.: yield=18%, d.e. =50%, (diastereomeric composition (−)/(+)(=25%/75%)).

Analysis of the mass balance of each diastereomer (crystal+mother liquors), shows that a second order asyininetric induced crystallization occurred during this optical resolution process. These results show the formation of the desired diastereomer at the expense of the unwanted one.

### EXAMPLE 1

Optical Resolution

To the free base racemic (S)-3-Methylamino-1-(2-thienyl)-1-propanol (1 g) dissolved in isopropanol (45 ml) was added the 2,3,4,6-di-O-isopropylidene-2-keto-L-gulonic acid (1.634 g) at room temperature and stirred for 4 hours.

The resulting salt is filtrated and dried under reduced pressure at 40° C. to yield 1.945 g. of a white solid (y=74%, d.e. =12%).

Optical purifications

The resulting solid of Example 1 (1.873 g, d.e. =12%) is suspended in isopropanol (82 ml) and stirred at room temperature for 69 hours.

The suspension is filtrated, dried under vacuum at 40° C. to yield 1.483 g of the diastereomeric salt (y=79%, d.e. =24%). The resulting solid (1.382 g, d.e. =24%) is suspended in isopropanol (159 ml) and stirred at room temperature for 16 hours. The suspension is filtrated, dried under vacuum at 40° C. to yield 0.803 g of the diastereomeric salt (y=58%, d.e. =78%)

### EXAMPLE 2

Racemization Procedure

To the diastereomeric salt (d.e. =75%) suspended in isopropanol (1 ml) was added HCl 1N (216 L). At that time solubilization of the salt occurred. The reaction mixture was stirred for 2 hours 30 minutes and concentrated under vacuum. The resulting solid has a d.e. =32% (CE analysis).

### EXAMPLE 3

Second Order Induced Crystallization

To the free base racemic (S)-3-Methylamino-1-(2-thienyl)-1-propanol (1 g) dissolved in isopropanol (31.3 ml) was added the 2,3,4,6-di-O-isopropylidene-2-keto-L-gulonic acid (1.634 g) at room temperature. The reaction mixture was heated to 40° C. and stirred for 66 hours at that temperature. After cooling to room temperature, the solid was filtrated and dried under reduced pressure at 40° C., to yield 1.993 g of a white solid (yield=76%, d.e. =76%).

Analysis of the mass balance of each diastereomeric (crystal+mother liquors), shows that a second order asymmetric induced crystallization occurred during this optical resolution process. These results show the formation of the desired diastereomer at the expense of the unwanted one.

The advantage of the present invention is found in its ability to prepare the desired product in high optical purity, with very little racemization, in short periods of time with resolution via diastereomeric salt formation as described above.

The synthesis of duloxetine is discussed in detail by Deeter, et al., in Tetrahedron Letters, 31(49), 7101-7104 (1990). Further synthetic Schemes 1 and 2 above, both of which are described in the prior art, provide enablement for making the racemic starting material for the current invention.

Briefly, as described in Scheme 2, the process described in Liu, H.; Hoff, B. H.; Anthonsen, T. Chirality, 12, 26 (2000), the (S)-chloroalcohol is derived from a Friedel-Crafts reaction of thiophenie and 3-chloropropionyl chloride. The chloroketone was reduced and the racemic alcohol is resolved enzymatically with immobilized Candida Antarctica Lipase B to give (S)-chloroalcohol in 35%. Yield (97% enantiomeric excess). See Liu et, al.

In Scheme 2 above, Wheeler, W. J.; Kuo, F. J. Labelled Compound Radiopharm., 36, 213 (1995) took a longer route to prepare “C-labeled duloxetine.” The chloroketone was reduced with a chiral borane reagent to give the (S)-chloroalcohol directly in 85% yield. Iodination and amination as before complete the synthesis of duloxetine.

A final route to either optically pure or racemic (S)-3-Methylamino-1-(2-thienyl)-1-propanol employs demethylation of the dimethylamino intermediate as shown below in Scheme 3 and 4. Precedent for the final route may be found in U.S. Pat. No. 5,225,585.

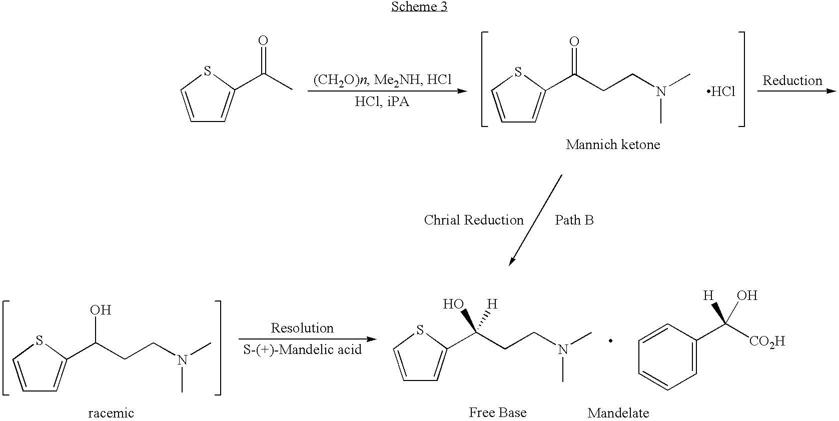

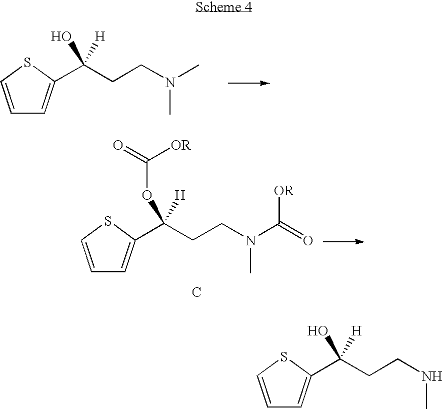

In Scheme 3 above, the starting material 2-acetylthiophene is converted to the Mannich ketone as described in Blicke, F. F.; Berckdhalter, S. H., J. Amer. Chem. Soc., 64, 451 (1941). The ketone is reduced to give racemic amino alcohol as described in Valenta, V.; Vilková, M.; Valchár, M.; Dobrovsky, K.; Polivka, 7, Collect Czech. Chem. Commun. 1991, 56, 1525; Jakobsen, P.; Kanstrup, A.; Lundbeck, J. M., Eur. Pat. Appl. EP 571,685, 1993 and Klosa, J., J Prakt. Chem., 1966, 34, 312 (Path A). Alternatively, the Mannich ketone can be reduced with chiral reducing agent to give the chiral S-amino alcohol directly (Path B).

Demethylation of the dimethylamino intermediate may be achieved by, for example, protecting the alcohol as a carbonate and using additional chloroformate to demethylate intermediate C above in Scheme 4 by organic chemistry methods shown in the art.

While the racemic compound I shown below as disclosed in Bopp, R. J. Kennedy, J. H., LC-GC, 6, 514 (1988), the resolution of this key intermediate has not been accomplished. The present invention achieves this resolution with high optical purity.

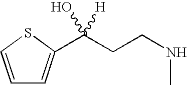

1. A process for preparing(S)-3-Methylamino-1-(2-thienyl)-1-propanol comprising:
resolving racemic —(S)-3-Methylamino-1-(2-thienyl)-1-propanol with (S)-(−)-2-pyrroliodone-5-carboxylic acid or 2,3,4,5-di-O-isopropylidine-2-keto-L-gulonic acid in a first organic solvent;
racemizing a stereomerically enriched mixture; and crystallizing (S)-3-Methylamino-1-(2-thienyl)-1-propanol by resolving racemic (S)-3-Methylamino-1-(2-thienyl)-1-propanol with 2,3,4,6-di-O-isopropylidine in a second organic solvent.

2. The process of claim 1 wherein the resolution of racemic (S)-3-Methylamino-1-(2-thienyl)-1-propanol is with 2,3,4,6-di-O-isopropylidine-2-keto-L-gulonic acid.

3. The process of any one of claims 1 or 2 wherein the first organic solvent is selected from isopropanol, tetrahydrofuran, acetone or ethyl acetate.

4. The process of any one of claims 1, or 2 wherein the first organic solvent is isopropanol.

5. The process of any one of claims 1 or 2 wherein the second organic solvent is selected from isopropanol, tetrahydrofuran, acetone or ethyl acetate.

6. The process of any one of claims 1 or 2 wherein the second organic solvent is isopropanol.
